(19) United States
(12) Patent Application Publication
(10) Pub. No.: US 2009/0092555 A1
(43) Pub. Date: Apr. 9, 2009

(54) Octameric Protein For Use In Bionanotechnology Applications

(76) Inventors: Vincent A. Fischetti, West Hempstead, NY (US); Daniel Craig Nelson, Rockville, MD (US); James Charles Whisstock, Victoria (AU); Ashley Maurice Buckle, Victoria (AU); Sheena McGowan, Victoria (AU)

Correspondence Address: FOX ROTHSCHILD LLP, 2000 MARKET STREET, 10th Floor, PHILADELPHIA, PA, 19103, US

(21) Appl. No.: 12/166,602

(22) Filed: Jul. 2, 2008

Related U.S. Application Data
(60) Provisional application No. 60/947,728, filed on Jul. 3, 2007.

(51) Int. Cl. A61K 49/14; C07K 7/64; A61K 38/12; A61L 27/28

(52) U.S. Cl. 424/9.34; 530/317; 514/11; 424/423; 324/693

(57) ABSTRACT

The present invention relates to purified multimeric proteins, comprising PlyCB monomers or functional fragments or variants thereof, which self-assemble into a ring like structure. The present invention also encompasses such multimeric proteins that comprise an integrin-binding sequence. The present invention further discloses contrast reagents, implant coatings, bone implants, and complexes for use in bionanotechnology applications, each of which comprise such multimeric proteins.

### CROSS REFERENCE TO RELATED APPLICATIONS

This application claims priority from U.S. Provisional Application No. 60/947,728, filed on Jul. 3, 2007, the disclosure of which is herein incorporated by reference.

### STATEMENT REGARDING FEDERALLY SPONSORED RESEARCH OR DEVELOPMENT

The research leading to the present invention was supported, in part, by a grant from the National Institutes of Health, All 1822 and by a grant from the Defense Advanced Research Projects Agency, DAAD19-01-1-0365. Accordingly, the United States Government has certain rights in the invention.

### INCORPORATION OF SEQUENCE LISTING

A computer readable form of the sequence listing, ‘P32869-A_USA_ST25.txtα (4,465 bytes), submitted via EFS-WEB and created on Jun. 26, 2008, is herein incorporated by reference.

### FIELD OF THE INVENTION

The invention relates to the identification and characterization of the PlyCB subunit of the PlyC lysin from the streptococcal bacteriophage C₁ and its use in bionanotechnology applications.

### BACKGROUND OF THE INVENTION

Bionanotechnology

Bionanotechnology is at the interface between physics, biology, chemistry, materials science, and engineering and is poised to be a key integrative technology of the 21st century. It is envisioned that bionanotechnology applications will include bio-circuits, bio-computers, artificial pores, drug delivery vehicles, biosensors, molecular motors, targeted therapeutics, artificial tissues, and medical imaging agents, to name a few. While expectations are extremely high, this field is still in its infancy and scientists are trying to develop the necessary tools that will be needed to realize many of these applications.

One of the most important short-term goals of bionanotechnology is to identify proteins that can act as a platform for further genetic or chemical modification. For example, -hemolysin, a heptameric pore-forming toxin secreted by Staphylococcus aureus, is being developed as a nanopore. Because this pore is of a proteinacious origin, it is possible to engineer residues in the lumen (i.e. change charge, etc.) by making genetic mutations, an advantage not afforded in a non-biological nanopore. These altered residues may serve various functions, such as binding heavy atoms for use in a biosensor. Furthermore, this protein is being studied for its potential use as a rapid DNA sequencing tool. Under this scenario, single stranded DNA is threaded through the -hemolysin pore in a lipid bilayer, a voltage is applied across the membrane, and changes in concurrent ion flow as DNA is threaded through the nanopore are used to distinguish purines from pyrimidines. (Ashkenasy et al., 2005). Another protein being developed for bionanotechnology applications is bacteriorhodopsin, based on its photoelectric, photochromic, and proton transport properties.

It is desirable that these platform or ‘scaffoldingα proteins include the following characteristics: the ability to self-assemble into the desired three-dimensional structure, display sidedness (i.e., the protein has different faces with different biochemical properties), contain a central pore and/or cage-like structure for delivery of functional agents, contain flexible linkers to add targeting moieties and/or functional agents, robust biochemical properties, and ability to scale-up/mass produce. While the proteins that are being currently developed for biotechnology applications likely contain some of these characteristics, it is believed that none of them contains all of these desired attributes. Accordingly, it is desirable to develop such a protein that contains as many of these attributes as possible.

Lysin and PlyCB

Bacteriophage cell wall hydrolases, or lysins, have recently been exploited for their bacteriolytic activity as an alternative to antibiotic therapy (Loessner, 2005; Fischetti, 2005). During a bacteriophage (or phage) infection cycle within a host organism, phage-directed proteins, called holins, are produced to perforate the bacterial membrane allowing the accumulating cytoplasmic lysins access to the cell wall (Young, 1992). The released lysins cleave covalent bonds in the peptidoglycan resulting in osmotic lysis of the bacterial cell and liberation of progeny phage. Appreciably, exogenous addition of purified lysins to susceptible Gram-positive bacteria also produces complete lysis, in the absence of bacteriophage (Schuch et al., 2002; Loeffler et al., 2001).

Other than PlyC, all Gram-positive cell wall hydrolyzing lysin family members described thus far are composed of a single polypeptide that has a modular design consisting of a well conserved catalytic domain and a cell wall binding domain. The catalytic domain is represented by one of four families of peptidoglycan hydrolases: N-acetylglucosaminidases, N-acetylmuramidases (lysozymes), N-acetylmuramoyl-L-alanine amidases, and endopeptidases (Moax & Molineux, 2004). In contrast, the cell wall binding domains are notably divergent and can distinguish discrete epitopes present within the cell wall, typically carbohydrates or teichoic acids, giving rise to the species- or strain-specific activity of a particular lysin.

The streptococcal C₁ bacteriophage lysin, now called PlyC for ββphage lysin from C₁,λλ was first described in 1957, when C₁ phage lysates were found to rapidly lyse cultures of groups A and C streptococci, despite the fact that the C₁ phage does not infect group A streptococci (Krause, 1957). Consequently, this enzyme has been used as a molecular tool for decades to isolate cell-wall-linked proteins and extract DNA from group A streptococci (Fischetti et al., 1985; Wheeler et al., 1980). It has been shown that the bacteriolytic properties of PlyC can protect mice from streptococcal challenge, suggesting a therapeutic use of the enzyme (Nelson et al., 2001).

Remarkably, genetic and biochemical analysis reveal that PlyC does not conform to the standard multi-domain, single polypeptide model that all other phage lysins adopt (Nelson et al., 2006). Instead, PlyC is a multimeric protein consisting of two separate gene products, PlyCA (50 kDa) and PlyCB (8 kDa). Based on biophysical studies, the catalytically active PlyC holoenzyme was found to be composed of eight PlyCB subunits for each PlyCA subunit (Id.). Inhibitor studies predicted the presence of an active-site cysteine and bioinformatic analysis revealed a cysteine-histidine aminopeptidase (CHAP) domain within PlyCA, which was confirmed by mutagenesis studies (Id.). While PlyCB possessed no catalytic activity, it was shown to be responsible for targeting PlyCA to the streptococcal cell wall (Id.). Moreover, PlyCB expressed in the absence of PlyCA retains the ability to form an octamer and this complex alone was able to direct streptococcal cell wall specific binding.

### SUMMARY OF THE INVENTION

The invention relates to purified PlyCB which is an octameric protein and a subunit along with PlyCA of the streptococcal C₁ bacteriophage lysin, PlyC. The octameric protein comprises eight polypeptides, each of which comprises the amino acid sequence of the PlyCB monomer or a functional fragment or variant thereof. The eight polypeptides self assemble into an octameric ring which defines a central pore of about 2 nm in diameter. In an embodiment of the invention, the octameric ring has a face that is neutral and another face that is positively charged. In one embodiment, each of the eight polypeptides comprise an N-terminal deletion variant of PlyCB (SEQ ID NO:2) or a functional fragment or variant thereof. In another embodiment, each of the eight polypeptides comprise the wild-type amino acid sequence of PlyCB (SEQ ID NO:1) or a functional fragment or variant thereof.

In one embodiment, the purified octameric protein includes eight polypeptides, each of which comprises an amino acid sequence at least 70%, 80%, 90%, or 95% identical to SEQ ID NO:2 capable of self-assembling into an octameric ring. In another embodiment, the purified octameric protein includes eight polypeptides, each of which comprises an amino acid sequence of SEQ ID NO:2 with 1 to 14, 1 to 10, 1 to 7, or 1 to 5 conservative amino acid substitutions.

In another embodiment of the present invention, the invention relates to a multimeric protein. The multimeric protein comprises about six to about twelve polypeptides, each of which comprises a sequence at least 70% identical to SEQ ID NO:2, and the polypeptides are capable of self assembling into a ring like structure.

In another aspect of the present invention, the purified octameric protein includes eight polypeptides and at least one of the polypeptides has a cellular-binding sequence of 3-9 amino acids. In one embodiment, the cellular-binding sequence is an integrin-binding sequence which is substituted for some or all of amino acids 13-17 of SEQ ID NO:2 or is covalently bound directly or via a linker sequence to the N-terminus of SEQ ID NO:2. The integrin-binding sequence is selected from the group consisting of RGD, DGR, REDV, VDER, NGR, RGN, NGRAHA, AHARGN, RCDVVV, VVVDCR, KGD, DGK, EDC, CDE, CDCRGDCFC, and CFCDGRCDC. In an embodiment of the invention, the integrin-binding sequence is RGD. In an alternate embodiment, each of the eight polypeptides of the octameric protein comprises SEQ ID NO:3, SEQ ID NO:4, SEQ ID NO:5, or SEQ ID NO:6.

In another embodiment, the invention relates to a contrast reagent for use with magnetic resonance imaging (MRI). The contrast reagent includes an octameric protein comprising eight polypeptides, each of the polypeptides comprises the amino acid sequence of SEQ ID NO:2, or a functional fragment or variant thereof, and a contrast agent bound to the octameric protein. In one embodiment, the contrast agent is a chelated lanthanide such as gadolinium. The contrast agent may be covalently bound to at least one of the eight polypeptides such as to a lysine residue via lysine-reactive 2-(4-isothiocyanatobenzyl)-diethylenetriamine-pentaacetic acid (DTPA-ITC). In another aspect of the present invention, the contrast reagent further includes a targeting moiety bound to said octameric protein such as a cellular-binding sequence, a monoclonal antibody or a metalloporphyrin.

In another embodiment, the present invention relates to an implant coating. The implant coating includes an octameric protein comprising eight polypeptides, each of the polypeptides comprises the amino acid sequence of SEQ ID NO:2, or a functional fragment or variant thereof capable of binding to hydroxyapatite, and an osteoblast binding agent bound to the octameric protein. In one embodiment, the osteoblast binding agent is a cellular-binding sequence such as an integrin-binding sequence. The implant coating may further include a bone-repair factor bound to the octameric protein. In another embodiment, the invention relates to a bone implant including an implant coated with hydroxyapatite and the implant coating discussed above.

In another embodiment, the present invention relates to a complex for use, for example, in bionanotechnology applications. The complex includes an octameric protein comprising eight polypeptides, each of the polypeptides comprises the amino acid sequence of SEQ ID NO:2, or a functional fragment or variant thereof, which self-assemble into an octameric ring having an outwardly facing surface and an inwardly facing surface, and a targeting agent and/or functional agent bound to the octameric protein. In an embodiment, the targeting agent is associated with the outwardly facing surface of the octameric protein and may be a cellular-binding sequence. Alternatively, the cellular-binding sequence may be covalently bound directly or via a linker sequence to the N-terminus of SEQ ID NO: 1 or SEQ ID NO:2. The functional agent may be associated with the outwardly or inwardly facing surfaces of the octameric protein.

In one embodiment of the present invention, the functional agent of the complex for use in bionanotechnology applications comprises a reporting agent such as the following: fluorophore, radionuclide, or quantum dot. In another embodiment, the functional agent of the complex may be a gold nanoparticle (nanogold) for use as a biocircuit. In another application of the complex, the functional agent can be a drug for targeted drug delivery.

In another embodiment, the present invention relates to a method of using the octameric protein to sequence DNA. The method includes providing a single-stranded nucleic acid comprising a sequence of nucleotides; providing an octameric protein comprising eight polypeptides, each of the polypeptides comprises SEQ ID NO:2 or a functional fragment or variant thereof, and the octameric protein is fixed in an electrically insulating membrane providing a channel there through; threading the single-stranded nucleic acid through the octameric protein; and measuring the changes in the conductance across the membrane by the sequential passage of the nucleotides.

### BRIEF DESCRIPTION OF THE DRAWINGS

FIG. 1A. Native PAGE of purified PlyC

FIG. 1B. An agarose gel been embedded with group A streptococci that was overlayed with an unstained native PAGE identical to FIG. 1A.

FIG. 1C. An SDS/PAGE of the purified PlyC used in FIG. 1A.

FIG. 2. Schematic of plyC gene showing three open reading frames, plyCB, lil, and plyCA.

FIG. 3. Gel filtration of PlyC (red), PlyCB (green), and molecular mass standards (blue). Inset: SDS/PAGE of the PlyC peak (red lane) and PlyCB peak (green lane).

FIG. 4. Top view of schematic diagram of the crystal structure of PlyCB showing eight identical monomers and a central pore.

FIG. 5A. Surface electrostatic potential map of the bottom and top surfaces of PlyCB.

FIG. 5B. Side view of schematic diagram of the crystal structure of PlyCB.

FIG. 6. A schematic diagram of a PlyCB monomer. The four-strands and two helicies are numbered sequentially from the N-terminus.

### DETAILED DESCRIPTION

### PlyCB Protein

The invention as disclosed and described herein, provides a purified PlyCB which is an octameric protein and a subunit of the streptococcal C₁ bacteriophage lysin, PlyC. PlyCB is useful as a ‘scaffoldingα protein for various bionanotechnology applications. The octameric protein comprises eight polypeptides, each of which comprises the amino acid sequence of the PlyCB monomer (SEQ ID NO:1) or a functional fragment or variant thereof.

PlyCB elutes from a gel filtration column at approximately 64 kDa and under 0.1% SDS conditions at approximately 8 kDa. (Nelson et al., 2006). Thus, in its natural state, PlyCB is an octameric protein comprising eight identical polypeptides. This octameric protein has a number of characteristics that make it an ideal platform protein for bionanotechnology applications.

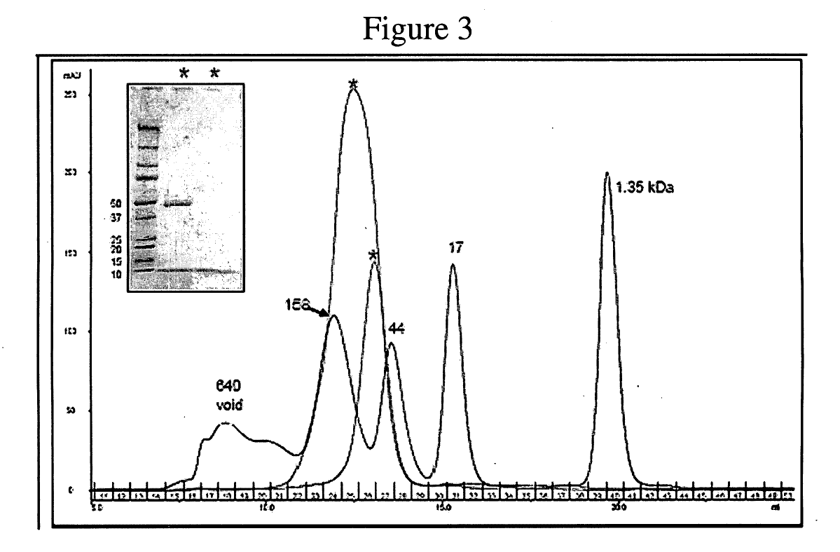

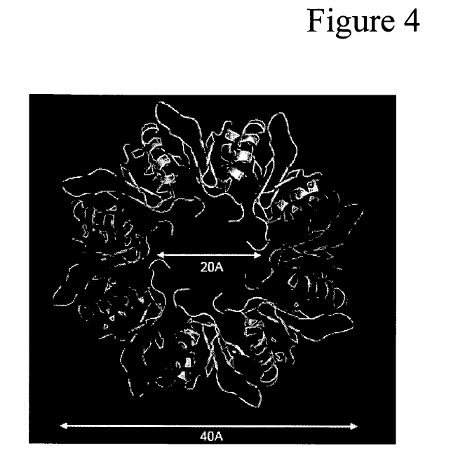

PlyCB self-assembles into an octameric ring structure even when expressed in the absence of PlyCλs other subunit, PlyCA. See FIGS. 3 and 4. PlyCB is denatured using urea into its 8 kDa monomers, and subsequent dialysis to remove the urea results in refolding of the 64 kDa octamer. Accordingly, a PlyCB octamer may be constructed of different monomers, and thus, the invention encompasses heteromers that comprise both variant monomers and wild-type monomers assembled into a functional octamer.

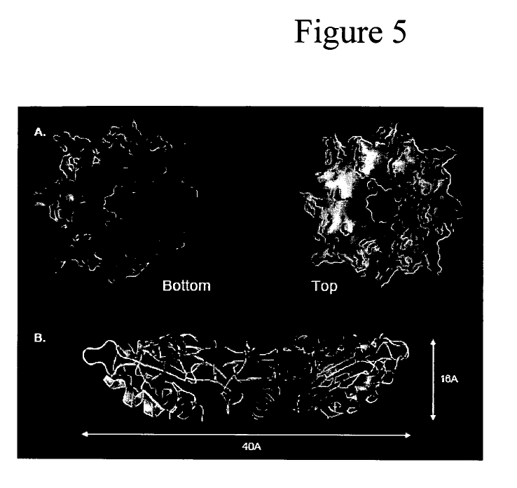

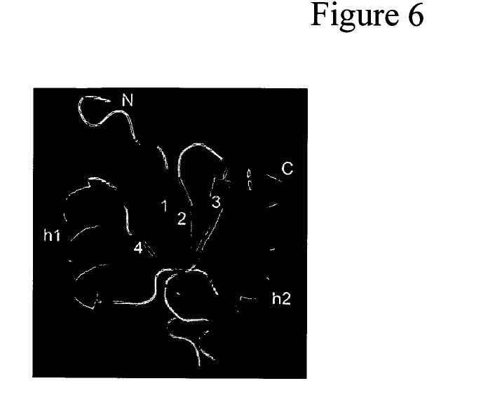

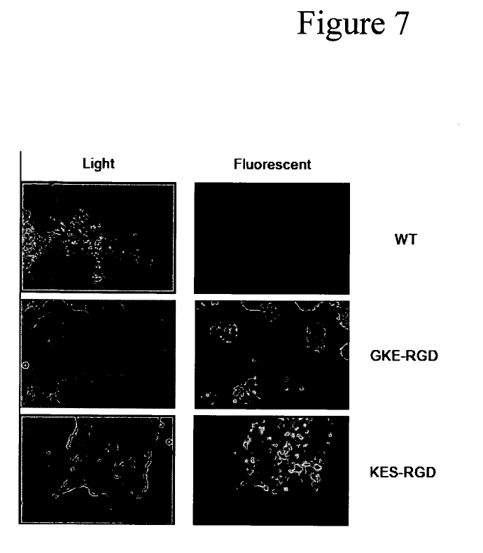

The first 8 N-terminal amino acids of PlyCB show no electron density in the crystal structure. See FIGS. 5 and 6. As confirmed by a deletion mutation of the first 8 N-terminal amino acids, this sequence does not appear to play a role in formation or stabilization of the octameric ring. Accordingly, this sequence can be readily modified, extended, or deleted depending on what is required for a particular bionanotechnology application. The invention, thus, encompasses an octameric protein comprising eight polypeptides, each of which comprises an 8 amino acid N-terminal deletion of PlyCB (SEQ ID NO:2) or a functional fragment or variant thereof. The present invention also encompasses heteromers that comprise both the 8 amino acid N-terminal deletion of PlyCB or a functional fragment or variant thereof, and the wild-type monomers assembled into a functional octamer.

The crystal structure of PlyCB further reveals a central pore approximately 2 nm in diameter. Accordingly, the invention encompasses using PlyCB as a nanopore. As used herein, the term ‘nanoporeαmeans a 1’ 100 nm hole in a thin membrane. This diameter is in the range of molecules and supramolecular structures that are biologically important at the cellular level. For instance, simple solutes such as amino acids, sugars and nucleotides are approximately 1 nm in diameter, the DNA double helix is ˜2 nm in diameter, and globular proteins such as those in blood plasma are 2-6 nm in diameter. A nanopore of the appropriate diameter may be used in an electrically insulating membrane as a single-molecule detector. The detection principle is based on monitoring the ionic current of an electrolyte solution passing through the nanopore as a voltage is applied across the membrane. For example, a nanopore may be used to sequence single-stranded DNA by passing the DNA molecule through the nanopore and observing changes in the current. A nanopore may also be used to separate single stranded and double stranded DNA in solution, to determine the length of polymers, to serve as biosensors for DNA hybridization and SNP detection, to identify proteins and viruses with single molecule resolution.

The central pore of PlyCB also gives PlyCB a cage-like structure. As discussed in more detail below, small molecules or peptides can be incorporated into the polypeptide sequence or bound to discrete locations of this cage-like structure for encapsulation, targeted delivery and release of therapeutic compounds. Release of the compounds may be accomplished by cleavage of a labile conjugation bond by pH changes or proteases up regulated in the particular microenvironment. As discussed below, this cage-like structure is also useful for use as a contrast reagent for magnetic resonance imaging (MRI).

PlyCB exhibits two distinct faces with different properties. X-ray crystallography reveals that PlyCB is a relatively flat protein (1.6 nm tall by 4 nm in diameter) which has two distinct faces that can interact with a substrate, analyte, or epitope. A surface electrostatic potential map (FIG. 5) shows a strong positive charge on one face and a neutral charge on the other face. The ‘sidednessα of the octameric protein may be used in bionanotechnology applications in which two distinct sides are advantageous such as a device which must interact with both a matrix and the environment. The present invention encompasses changes to the surface electrostatic potential of PlyCB by deleting, inserting or substituting amino acids in one or more of the PlyCB monomers. For example, the positively charged C-terminal amino acids Lysine-Lysine may be replace with a negatively charged glutamic acid.

The PlyCB octamer is very stable. It retains its three-dimensional structure upon heating to 50° C. and can be frozen, lyophilized, or stored at 4° C. in excess of one year without any deleterious effects. PlyCB is not affected by large changes in pH or exposure to high ionic environments or non-ionic detergents. Only strong ionic detergents, such as SDS, have been shown to disrupt the PlyCB octamer. Furthermore, mutations or deletion of the N-terminal region does not affect the structural integrity of the octamer. Likewise, most mutations or modifications to the external loop between -strand 1 and 2 (see FIG. 6) do not affect the structural integrity of the octamer. The multiple intermolecular contacts between the -helices of neighboring PlyCB monomers are thought to provide considerable stability to the octamer. As used herein, the term ‘octameric proteins refers to the purified PlyCB protein which comprises eight polypeptides each of which comprises the amino acid sequence of the PlyCB monomer (SEQ ID NO:1). The term ‘octameric proteins also encompasses functional equivalents of PlyCB comprising eight polypeptides of functional fragments or variants of the PlyCB monomer which are capable of self-assembling into an octameric ring. For example, the eight polypeptides may comprise an N-terminal deletion mutant of the first 8 amino acids (SEQ ID NO:2). The term ‘octameric proteinα also encompasses heteromers that comprise any combination of wild-type monomers, fragments of the wild-type monomers, or variants of the wild-type monomers which are capable of self-assembling into an octameric ring.

The present invention also encompasses other ‘multimeric proteinsα. As used herein, the term ‘multimeric proteins refers to a protein comprising about 6 to about 12 polypeptides each of which comprises the amino acid sequence of the PlyCB monomer (SEQ ID NO:1), or fragments or variants thereof, which are capable of self-assembling into a ring-like structure.

The term ‘polypeptideα includes any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. The polypeptides of the present invention include natural peptides, recombinant peptides, synthetic peptides, or a combination thereof.

In one embodiment, non-native amino acids may be introduced into the polypeptides of the present invention to add unique physiochemical and biological properties to the octameric protein. For example, because the PlyCB monomer (SEQ ID NO:1) only contains a single methionine (Met69), which can be changed to an alanine (M69A) without any loss of functionality or octamer formation, a methionone may be genetically introduced or an existing residue may be changed into a methionine through point mutagenesis. Using this approach, in combination with a methionine auxotrophic strain and minimal media, any methionine analog such as homoallylglycine (Hag) can be specifically inserted or substituted into the PlyCB monomer. (van Hest and Tirrell, 1998). The present invention also encompasses the use of other modified or unusual amino acids to achieve a desired physiochemical or biological property such as 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2λ-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine (sarcosine), N-methylisoleucine, 6-N-methyllysine, N-methylvaline, norvaline, norleucine and ornithine.

In another embodiment, custom tRNAs:aminoacyl-tRNA-synthase pairs may be used to incorporate up to 30 unnatural amino acids that provide unique reactive groups, photoreactive side chains, post-translational modifications, biophysical probes, metal chelators, etc. (Xie and Schultz, 2006).

The polypeptides of the invention also include ‘functional fragmentsα of polypeptides as the term is used herein. A ‘functional fragments of a polypeptide includes a portion of the polypeptide that retains its functional characteristics, for example, in the context of the ‘octameric proteins of the present invention, its ability to self-assemble with seven such other fragments or other PlyCB monomers into an octameric ring-like structure.

The polypeptides of the invention include ‘functional variantsα of polypeptides as the term is used herein. A ‘functional variantα of a polypeptide includes a variant of the polypeptide that retains its functional characteristics, for example, in the context of the ‘octameric proteinα of the present invention, its ability to self-assemble with seven such other variants or other PlyCB monomers into an octameric ring-like structure. A ‘variants further refers to variations in the sequence of the polypeptides that may arise naturally or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules. Substitutions may be designed based on, for example, the model of Dayhoff et al. (1978). These modifications can result in changes in the amino acid sequence, provide silent mutations, modify a restriction site, or provide other specific mutations.

In one embodiment, the present invention relates to a octameric protein including eight polypeptides, each of the eight polypeptides comprise an 8 amino acid N-terminal deletion variant of PlyCB (SEQ ID NO:2) or a functional fragment or variant thereof. As discussed in the Examples section below, the 8 amino acid N-terminal deletion variant of PlyCB (SEQ ID NO:2) retains its three-dimensional structure as evidenced by gel filtration and the retention of its ability to bind to streptococcal cells.

In another embodiment, the purified octameric protein includes eight polypeptides, one or more of which comprises an amino acid sequence at least about 50%, 65%, 70%, 75%, 80%, 85%, 90%, 95%, 98%, 99% identical to the amino acid sequence of SEQ ID NO:1 or SEQ ID NO:2 capable of self-assembling into an octameric ring. To determine the percent identity of two amino acid sequences or two nucleic acid sequences, the sequences are aligned for optimal comparison purposes (i.e., gaps can be introduced in the sequence of a first amino acid for optimal alignment with a second amino acid sequence). The amino acid residues at corresponding positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. One, non-limiting example of a mathematical algorithm utilized for the comparison of two sequences is the algorithm of Karlin and Altschul (1990), modified as in Karlin and Altschul (1993). Such an algorithm is incorporated into the NBLAST and XBLAST program of Altschul et al. (1990). BLAST nucleotide searches can be performed with the NBLAST program, score=100, wordlength=12 to obtain nucleotide sequences homologous to a nucleic acid molecules of the invention. The BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al. (1997). Alternatively, PSI-Blast can be used to perform an iterated search which detects distant relationships between molecules (Id.). When utilizing BLAST, Gapped BLAST and PSI-Blast programs, the default parameters of the respective programs (i.e., XBLAST and NBLAST program can be used (see, HTTP://WWW.NCBI.NLM.NIH.GOV). Another preferred, non-limiting example of a mathematical algorithm utilized for the comparison of sequences is the algorithm of Myers and Miller (1988). Such an algorithm is incorporated into the ALIGN program (version 2.0) which is part of the GCG sequence alignment software package. When utilizing the ALIGN program for comparing amino acid sequences of a PAM 120 weight residue table, a gap length penalty of 12 and a gap penalty of 4 can be used. In an alternate embodiment, alignments can be obtained using the NA-MULTIPLE-ALIGNMENT 1.0 program, using a GapWeight of 5 and a GapLengthWeight of 1.

In another embodiment, the purified octameric protein includes eight polypeptides, one or more of which comprises an amino acid sequence of SEQ ID NO:2 with 1 to 14, 1 to 10, 1 to 7, or 1 to 5 conservative amino acid substitutions. Examples of conservative substitutions include substitution of amino acids that do not alter the secondary and/or tertiary structure of PlyCB. Additional examples include substituting one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are known in the art.

PlyCB is easily modified and mass produced. As discussed in the Examples, PlyCB is cloned into a simple arabinose-inducible E. Coli expression system (pBAD24 vector). Current yields without optimization are approximately 50 mg/L of culture. PlyCB is easily purified by one-step purification process using a hydroxyapatite affinity column.

Cellular-Binding Mutants

In another aspect of the present invention, the purified octameric protein includes eight polypeptides and at least one of the polypeptides has a cellular-binding sequence of approximately 3-9 amino acids. As used herein, the term ‘cellular-binding sequences refers to polypeptide sequences that direct binding to cellular surface proteins.

In one embodiment, the cellular-binding sequence is an integrin-binding sequences. Integrins are a class of cellular receptors responsible for cell-matrix adhesion. As a result, integrins are involved in angiogenesis, cancer, thrombosis, and osteoporosis and may serve a key role in human disease. Integrins are multimeric proteins consisting of one of 18-subunits and one of eight-subunits, forming 24 known heterodimers. As used herein, ‘integrin-binding sequencesα refer to polypeptide sequences that can bind to one or more integrins. One particular tri-peptide sequence Arg-Gly-Asp, or ‘RGDα is sufficient to direct binding to several of the predominant integrins including 3, 5, and 51. Other sequences such as DGR, REDV, NGR, NGRAHA, RCDVVV, KGD, EDC, and CDCRGDCFC have also been shown to bind to these integrins. Accordingly, the present invention encompasses the use of all such integrin-binding sequences including the inverse of such integrin-binding sequences.

3 is a vitronectin and fibronectin receptor involved in angiogenesis and bone resorption. Likewise, 5 and 51 are alternate cellular receptors implicated in angiogenesis. Induction of angiogenesis by tumors or cytokines has been shown to up-regulate these integrins. Consequently, the octameric protein of the present invention, having the ability to bind these integrins, may be used as a therapeutic (e.g., by delivery of cytotoxic compounds to tumor tissues), diagnostic, or for imaging purposes (e.g, by binding paramagnetic particles).

In another embodiment, the cellular-binding sequence is a heparin-binding sequence. The heparin-binding sequences are typically polycationic and bind to negatively charged glycosaminoglycans associated with proteoglycans found in the cell membrane. Heparin-binding sequences include KRSR and FHRRIKA. Accordingly, the present invention encompasses the use of all such heparin-binding sequences including the inverse of such heparin-binding sequences.

The present invention also encompasses the use of other cell-binding sequences, including the inverse of such sequences, such as GFOGER, derived from collagen, and IKVAV and YIGSR, derived from laminin.

In one embodiment, the cellular-binding sequence is substituted for some or all of amino acids located in the loop structure between -strands 1 and 2 (see FIG. 6) of at least one of the peptides that forms the octameric protein encompassing the sequence D₂₁G₂₂K₂₃E₂₄S₂₅ of SEQ ID NO:1. With respect to SEQ ID NO:2, these amino acids are at positions 13-17. For example, in an embodiment of the present invention, at least one of the eight polypeptides of the octameric protein comprises the amino acid sequence of SEQ ID NO:3 (RGD substituted for amino acids 22-24 of SEQ ID NO:1), SEQ ID NO:4 (RGD substituted for amino acids 14-16 of SEQ ID NO:2), SEQ ID NO:5 (RGD substituted for amino acids 23-25 of SEQ ID NO:1), or SEQ ID NO:6 (RGD substituted for amino acids 15-17 of SEQ ID NO:2).

In an alternative embodiment, the cellular-binding sequence of about 3-9 amino acids is covalently bound directly or via a linker sequence to the N-terminus of SEQ ID NO:1 or SEQ ID NO:2 to at least one of the peptides that forms the octameric protein.

Contrast Reagent

In another embodiment, the invention relates to a contrast reagent for use with magnetic resonance imaging (MRI). The contrast reagent includes an octameric protein comprising eight polypeptides, each of the polypeptides comprises the amino acid sequence of SEQ ID NO:2, or a functional fragment or variant thereof, and a contrast agent bound to the octameric protein.

The term ‘magnetic resonance imaging (MRI)α refers to a method to render images of the inside of an object. It is primarily used in medical imaging to demonstrate pathological or other physiological alterations of living tissues. MRI scanners can generate multiple two-dimensional cross-sections (slices) of tissue and three-dimensional reconstructions. MRI uses non-ionizing radio frequency (RF) signals to acquire its images and is best suited for non-calcified tissue. MRI may be enhanced by the use of contrast agents that have paramagnetic properties.

The contrast agents of the present invention may be any molecule that can be coupled to the octameric protein that is capable of increasing the contrast of the images from a MRI scanner. In one embodiment, the contrast agent is a paramagnetic agent such as a lanthanide (e.g., a gadolinium compound). Gadolinium-enhanced tissues and fluids are known to appear extremely bright on T₁-weighted images. This provides high sensitivity for detection of vascular tissues (e.g. tumors) and permits assessment of brain perfusion (e.g. in stroke). Gadolinium compounds on the market that could be used with the present invention include Magnevist (gadopentetate dimeglumine), Ominiscan (gadodiamide), OptiMARK (gadoversetamide), MultiHance (gabobenate dimeglumine) and ProHance (gadoteridol). The contrast agents may also be a superparamagnetic agent (e.g. iron oxide nanoparticles). These agents appear very dark on T₂-weighted images and may be used for liver imaging—normal liver tissue retains the agent, but abnormal areas (e.g. scars, tumors) do not. Ferrioxamine methanesulfonate which is currently being tested as a contrast agent for the kidneys, ureters and bladder may also be used.

The present invention also encompasses contrast reagents in which the contrast agent is covalently bound to at least one of the polypeptides of the octameric protein. In a particular embodiment, the contrast agent is bound via lysine-reactive 2-(4-isothiocyanatobenzyl)-diethylenetriamine-pentaacetic acid (DTPA-ITC).

The contrast reagent of the present invention may also encompass a targeting moiety. As used herein, the ‘targeting moietyα may be any substance such as a peptide, antibody or small chemical molecule that directs the contrast reagent to particular cell types or tissues.

For example, the targeting moiety may be a cellular-binding sequence of 3-9 amino acids that is incorporated into the sequence for at least one of the polypeptides of the octameric protein as discussed above. In particular, the cellular-binding sequence of 3-9 amino acids may be substituted for some or all of amino acids 13-17 of SEQ ID NO:2. Alternatively, the cellular-binding sequence may be covalently bound directly or via a linker sequence to the N-terminus of SEQ ID NO:1 or SEQ ID NO:2. The cellular-binding sequence may be an integrin-binding sequence selected from the group consisting of RGD, DGR, REDV, VDER, NGR, RGN, NGRAHA, AHARGN, RCDVVV, VVVDCR, KGD, DGK, EDC, CDE, CDCRGDCFC, and CFCDGRCDC.

By way of another example, the targeting moiety can be a moiety designed to target tumors, either specifically or nonspecifically. One such targeting moiety is a monoclonal antibody designed to target specific tumor types such as adenocarcinoma of the colon. Another targeting moiety is metalloporphyrins which exhibit affinity for many tumor types including carcinoma, sarcoma, neuroblastoma, melanoma and lymphoma.

Implant Coating/Implant

In another embodiment, the present invention relates to an implant coating. The implant coating includes an octameric protein comprising eight polypeptides, each of the polypeptides comprises the amino acid sequence of SEQ ID NO:2, or a functional fragment or variant thereof capable of binding to hydroxyapatite, and an osteoblast binding agent bound to the octameric protein.

Frequently implant materials are not compatible with osteoblasts responsible for bone formation, but rather they promote the formation of undesirable soft connective tissue. Fibrous soft tissue, as opposed to hard boney tissue, has been shown to improperly fix orthopedic implants into surrounding bone which leads to loosening under physiological loading conditions and eventual implant failure. The implant coating of the present inventions increases the adsorption of osteoblasts on the surface of the implant to improve bone formation.

The term ‘hydroxyapatiteα as used herein refers to hydroxylapatite or hydroxyapatite, a calcium mineral. ‘Hydroxyapatiteα is a naturally occurring form of calcium apatite with the formula Ca₅(PO₄)₃(OH), but is usually written Ca₁₀(PO₄)₆(OH)₂ to denote that the crystal unit cell comprises two molecules. The OH⁻ ion can be replaced by other anionic groups such as fluoride, chloride or carbonate. ‘Hydroxyapatiteα also encompasses other calcium apatites which will support bone ingrowth and osseointegration.

The term ‘osteoblast binding agents as used herein encompasses all peptides, antibodies and small chemical molecules capable of binding to osteoblasts. For example, the osteoblast binding agent may be a cellular-binding sequence of 3-9 amino acids that is incorporated into the sequence for at least one of the polypeptides of the octameric protein as discussed above. In particular, the cellular-binding sequence of 3-9 amino acids may be substituted for some or all of amino acids 13-17 of SEQ ID NO:2. Alternatively, the cellular-binding sequence may be covalently bound directly or via a linker sequence to the N-terminus of SEQ ID NO:1 or SEQ ID NO:2. The cellular-binding sequence may be an integrin-binding sequence selected from the group consisting of RGD, DGR, REDV, VDER, NGR, RGN, NGRAHA, AHARGN, RCDVVV, VVVDCR, KGD, DGK, EDC, CDE, CDCRGDCFC, and CFCDGRCDC.

The implant coating of the present invention may also include bone-repair factors. As used herein, ‘bone-repair factorsα refers to all the factors involved in optimal bone regeneration. Such factors include proinflammatory cytokines (e.g., interleukin-1, interleukin-6 and tumor necrosis factor-), growth and differentiation factors (e.g., transforming growth factor-, bone morphogenetic proteins, platelet derived growth factor, fibroblast growth factor, insulin-like growth factor), metalloproteinases, and angiogenic factors. Such factors may be cross-linked to the octameric protein by techniques known in the art.

In another embodiment, the invention relates to a bone implant including an implant coated with hydroxyapatite, and an implant coating which includes an octameric protein comprising eight polypeptides, each of the polypeptides comprises the amino acid sequence of SEQ ID NO:2, or a functional fragment or variant thereof capable of binding to hydroxyapatite, and an osteoblast binding agent bound to the octameric protein. The osteoblast binding agent is as described above. The implant coating may also include bone-repair factors as described above.

The term ‘bone implants or ‘implants as used herein refers to material used to replace damaged or troubled bones or joints such as hip, knee, shoulder and elbow. The term ‘bone implants or ‘implants also refers to implants for dental use. The term also encompasses bone plates, bone screws and other fasteners used in orthopedic procedures. Such implants may be made of any biocompatible material such as titanium.

In use, prior to implantation, the bone implant is coated with hydroxyapatite by procedures known in the art. The hydroxyapatite coated implant is then coated with the implant coating of the present invention. While not wishing to be bound to any one particular theory, it is believed that the strong positively charged face of the octameric protein ionically binds to the hydroxyapatite. After implantation, the octameric proteins osteoblast binding sites attract osteoblasts to the surface of the implant. For example, an integrin-binding sequence substituted for some or all of amino acids 13-17 of SEQ ID NO:2 can bind to integrins on the cell surface of the osteoblasts. The osteoblasts are thus able to colonize on the implant surface and synthesize new bone tissue filling any gaps between the implant and juxtaposed bone.

Bionanotechnology Complex

In another embodiment, the present invention relates to a complex for use, for example, in bionanotechnology applications. The complex includes an octameric protein comprising eight polypeptides, each of the polypeptides comprises the amino acid sequence of SEQ ID NO:2, or a functional fragment or variant thereof, which self-assemble into an octameric ring having an outwardly facing surface, an inwardly facing surface, and an interstitial area, and a targeting agent and/or functional agent bound to the octameric protein.

The term ‘inwardly facing surfaced refers to the surface facing the pore of the octameric protein. The term ‘outwardly facing surfaced refers to the exterior of the cage-like octameric protein. The ‘interstitial areas is the area between the polypeptide monomers which interact to form the stable octamer.

The term ‘targeting agents encompasses all peptides, antibodies and small chemical molecules capable of targeting the complex to a particular location. For example, the ‘targeting agents may target the complex to a particular cell-type or tissue. More than one ‘targeting agents may be used on a particular octameric protein. In one embodiment, the ‘targeting agents is prepared by genetically or synthetically incorporating ligands onto the outwardly facing surface of the octameric protein.

In one embodiment, the targeting agent is one of the cellular-binding sequences discussed above. The cellular-binding sequence may be substituted for some or all of amino acids 13-17 of SEQ ID NO:2. Alternatively, the cellular-binding sequence may be covalently bound directly or via a linker sequence to the N-terminus of SEQ ID NO:1 or SEQ ID NO:2. In one example, the cellular-binding sequence is an integrin-binding sequences selected from the group consisting of RGD, DGR, REDV, VDER, NGR, RGN, NGRAHA, AHARGN, RCDVVV, VVVDCR, KGD, DGK, EDC, CDE, CDCRGDCFC, and CFCDGRCDC.

The term ‘functional agents encompasses all peptides, antibodies and small chemical molecules that serve some function other than targeting the complex. The functional agent may be associated with the outwardly or inwardly facing surfaces, or interstitial area of the octameric protein. Such functional agents include small molecules, imaging agents, gold nanoparticles, fluorophores, carbohydrates, nucleic acids and peptides. As with the ‘targeting agents the octameric nature of the complex allows for the addition of more than one ‘functional agent.α In one embodiment, the functional agent is bound to the inwardly facing surface or interstitial area of the octameric protein. For example, a cysteine residue genetically introduced into one of the polypeptides of the octameric protein present a reactive thiol group in the assembled octameric cage-like structure. Medically relevant small molecules such as therapeutics (e.g., anticancer drugs) and imaging agents can be chemically attached to these reactive thiol groups. (Flenniken et al., 2005).

In one embodiment, the functional agent is a reporting agent. The term ‘reporting agents refers to any peptide or small chemical molecules that can be used to identify the complex. In a particular embodiment, the reporting agent may contain a fluorophore, which is a functional group in a molecule which will absorb energy of a specific wavelength and re-emit energy at a different (but equally specific) wavelength. A common fluorophore is fluorescein isothiocyanate, a reactive derivative of fluorescein. Other common fluorophores are derivatives of rhodamine, coumarin and cyanine.

In another embodiment, the reporting agent is a quantum dot. As used herein, a ‘quantum dote is a semiconductor nanostructure that confines the motion of conduction band electrons, valence band holes, or excitons (bound pairs of conduction band electrons and valence band holes) in all three spatial directions. Quantum dots can be used in replace of traditional fluorophores as they are brighter (owing to the high quantum yield) as well as their stability (much less photodestruction). In use with the complex of the present invention, the quantum dot is bound to the complex and the complex is targeted to a particular cell type of tissue for imaging.

In another embodiment, the reporting agent is a radionuclide. As used herein a ‘radionuclideα is an atom that omits ionizing radiation. In use with the complex of the present invention, radionuclides emitting gamma rays are incorporated into the complex and can provide diagnostic information about an organismλs internal anatomy and the functioning of specific organs using single photon emission computed tomography (SPECT) and positron emission tomography (PET) scanning. Moreover, the radionuclides may be targeted to specific cell types and tissues as discussed above. Radionuclides may also be used in the present invention along with integrin-binding sequences to target tumors for treatment.

In another embodiment of the present invention, the functional agent is a gold nanoparticle or nanogold. The term ‘gold nanoparticleα or ‘nanogoldα refers to a particle of Au having at least one diameter less than 100 nm. In a preferred embodiment, the gold nanoparticle is spherical and about 2 nm or less in diameter and able to fit within the pore of the octameric protein. The gold nanoparticle may be bound to the octameric protein via a reactive thiol group introduced by the incorporation of cysteine residues into at least one of the polypeptides of the octameric protein. Other soft metals such as zinc may be substituted for the gold nanoparticle. The PlyCB-gold nanoparticle complex may be formed into 2D crystals and bound to a substrate to form an array of PlyCB-gold nanoparticle complexes. These arrays may be used in bioelectronic or photonic devices.

In another embodiment of the present invention, the functional agent is a drug. As used herein, the term ‘drugs means any chemical or biological substance, synthetic or non-synthetic, intended for use in the diagnosis, cure, mitigation, treatment, or prevention of disease. The complex comprising the drug may be targeted to specific cells or tissues in an organism such as by incorporating integrin-binding sequences into at least one of the polypeptides of the octameric protein as discussed above. In one embodiment, the drug is bound to the inwardly facing surface of the octameric protein and thus is sequestered until directed to be released. Accordingly, during administration and clearance the encapsulated drug remains invisible to the exterior environment and therefore toxicity and side effects may be reduced.

In one embodiment, the complex is used for oral cavity applications. For example, the complex may be targeted to oral pathogens (e.g., Streptococcus mutans) through a specific binding sequence or antibody. The complex may further contain a conjugated antimicrobial such as lactoferrin, defensin, or histatin.

Release of the drug from the octameric protein may be accomplished in any number of ways known in the art. For example, the drug may be bound to PlyCB via a cross-linker and release of the drug is achieved by cleavage of one of the cross-linker bonds by pH changes or by thiol cleavage. Linkers susceptible to such cleavage are known in the art such as diazobenzyme, diimide esters, maleimides, and pyridyl disulfide. For example, in a particular embodiment, the drug is an anticancer drug such as doxorubicin. Doxorubicin may be chemically attached to the octameric protein via reactive thiol group introduced by the incorporation of cysteine residues into at least one of the polypeptides of the octameric protein. (Flenniken et al., 2005).

Alternatively or additionally, linkers with specific proteolytic cleavage sites, such as matrix metalloproteinase (MMP)-2 cleavable peptides, may be used to link a drug to PlyCB for targeted delivery. Up regulation of the MMP-2 protease by tumor cells cleaves the MMP-2 cleavable peptides releasing the drug from the PlyCB octameric ring.

Nucleotide Sequencing

The present invention further relates to a method of using the octameric protein to sequence DNA. The method includes providing a single-stranded nucleic acid comprising a sequence of nucleotides; providing an octameric protein comprising eight polypeptides, wherein each of the polypeptides comprises SEQ ID NO:2 or a functional fragment or variant thereof, and the octameric protein is fixed in an electrically insulating membrane providing a channel there through; threading the single-stranded nucleic acid through the octameric protein; and measuring the changes in the conductance across the membrane by the sequential passage of the nucleotides.

As used herein, the term ‘single-stranded nucleic acidα comprises single-stranded DNA, cDNA, RNA, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases.

As used herein, the term ‘electrically insulating membranes means a membrane such that when a voltage is placed across an insulator, no charge or current flows. ‘Electrically insulating membranesα encompass both biological membranes such as a lipid bilayer and sold-state membranes such as those made from silicon compounds (e.g., Si₃N₄).

### EXAMPLES

### Example 1 Cloning, Expression, Purification and Sequencing of PlyCB

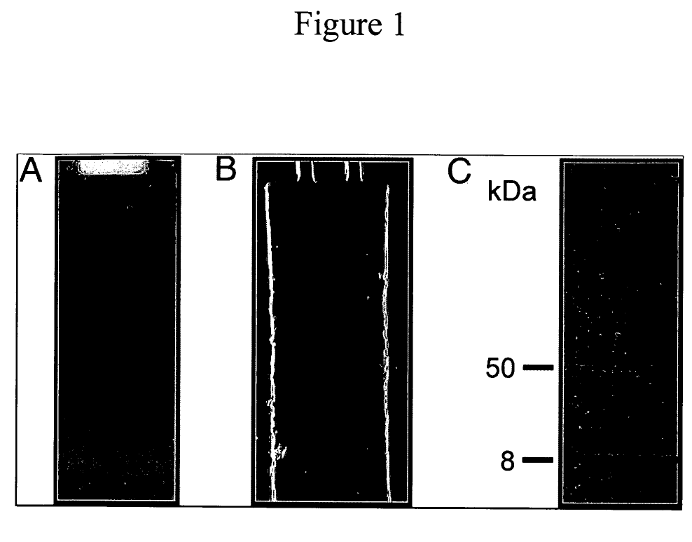

PlyC Consists of a Heavy Chain (PlyCA) and a Light Chain (PlyCB)—PlyC purified from C₁ bacteriophage lysates behaves as a homogeneous protein on native-gel electrophoresis (FIG. 1A). Moreover, this protein band is responsible for the lytic activity, as observed by formation of a clearing zone on an overlay of streptococci-embedded agarose (FIG. 1B). However, SDS/PAGE analysis of the same material on a 4‘20% gradient gel revealed the presence of two bands (FIG. 1C), an approximately 50-kDa heavy chain, termed PlyCA, and an approximately 8-kDa light chain, termed PlyCB, neither of which displayed lytic activity on overlay. N-terminal sequencing of the PlyC heavy and light chains results in two unique sequences (SKKYTQQQYE and SKINVNVENV, respectively), and sequencing of the native band results in a dual sequence, which corresponds exactly to both chains.

Cloning of PlyC Genes—C₁ bacteriophage genome is partially digested with Tsp509I and the fragments are ligated into a pBAD24 vector. Screening the resulting Escherichia coli expression library reveals a single clone (pBAD24::plyC) that encodes lytic activity toward group A streptococci (Schuch et al., 2002).

Expression and Purification of the PlyC Clone—PlyC is induced with 0.5% arabinose overnight from XL1-blue/pBAD24::plyC cultures. Cells are washed in 20 mM phosphate buffer at pH 7.0 and are lysed with 20% wt/vol chloroform to yield crude PlyC. Alternatively, group C streptococci are infected with the C1 bacteriophage as previously described (Nelson et al., 2001), and phage-produced PlyC are isolated from the crude lysate. Purification of the phage-derived enzyme and the recombinant enzyme are identical to previously described methods (Nelson et al., 2001). Purified enzymes are routinely stored in PBS at 4° C. and are stable for several months. Purification of the individually expressed heavy and light chains use the same column-chromatography methods as the native enzyme. Native and SDS/PAGE analyses are performed according to the method of Schagger and von Jagow 1987, and blotting to poly(vinylidene difluoride) membranes is according to Matsudaira, 1987. N-terminal sequencing is performed at The Rockefeller University Proteomics Resource Center. PlyCB, native PlyC, and cross-linked PlyC samples are subjected to analytical gel filtration for size estimation on a Superose 12 column (Amersham Pharmacia Biosciences) calibrated with gel-filtration standards (Bio-Rad).

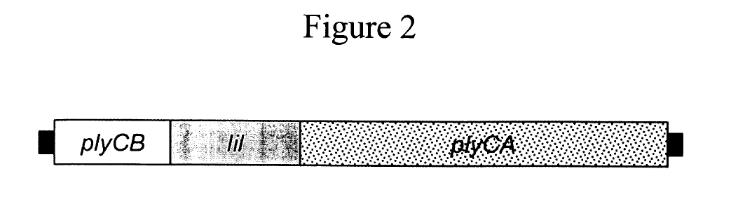

Sequencing the PlyC Genes—The purified, recombinant PlyC has identical properties on column chromatography and native and SDS/PAGE as compared with PlyC purified from phage lysates. The plyC clone contains a 2.2-kb insert comprising three putative ORFs in addition to approximately 100 bp of a noncoding sequence flanking the 5λ and 3λ ends of the insert (FIG. 2). These genes correspond to ORFs 9, 10, and 11 of the recently sequenced C₁ phage genome (Nelson et al., 2003). The first gene of the 2.2-kb insert, hereafter referred to as plyCB, encodes a 72-aa polypeptide that matches the N-terminal sequence of PlyCB without the N-terminal methionine (most likely removed by methionine aminopeptidases present in the E. coli cytoplasm). SEQ ID NO:1 comprises the 71-aa polypeptide without the N-terminal methionine. The 7.858-kDa predicted mass of the plyCB gene product approximates the observed size of PlyCB by SDS/PAGE (FIG. 1C). A position-specific iterative (PSI)-BLAST search reveals no significant matches for this protein in any database.

The second gene of the plyC clone encodes a putative 105-aa protein, which has significant homology (E value better than threshold) to 31 endonucleases, most of them belonging to the β’HNHλλ endonuclease family, such as those for Streptococcus agalactiae prophage _Sa2 (ANN00738), Lactococcus phage bIL170 (AAC27227), and Vibriophage VpV262 (AAM28379). HNH endonucleases are known to embed themselves within group I introns and confer mobility to the host intervening sequence (Chevalier & Stoddard, 2001). Because of the unique position of the putative endonuclease gene between plyCB and the third ORF, this region is named lil, for lysin intergenic locus.

The third gene of the 2.2-kb insert, hereafter referred to as plyCA, encodes a 465-aa polypeptide with a predicted size of 50.333 kDa, matching both the size and N-terminal sequence of PlyCA. A PSI-BLAST search indicates moderate homology to putative minor structural proteins from the Streptococcus thermophilus phage Sfi11 (AAC34413) and S. agalactiae prophage SA03 (ABA46334) as well as a putative tail protein of the Streptococcus pyogenes prophage 315.5 (AAM79918).

Cloning, Expression and Purification of PlyCB—Plasmid DNA is purified from XL1-blue/pBAD24::plyC, and the plyCB ORF is amplified by PCR using primers Start (5λ-GTA CCC GGG GAA GTA ATT TCC ATT CTT GAA-3λ) and Light-R (5λ-CCC AAG CTT TTA CTT TTT CAT AGC CTT TCT-3λ). The forward primer contains a SmaI site, and the reverse primer contains a HindIII site and a stop codon. PCR products are digested by SmaI and HindIII and are ligated into a SmaI/HindIII-digested arabinose-inducible expression pBAD24 (Amp^{r}) vector. Finally, XL1-blue cells are cotransformed with pBAD24::plyCB and cells displaying a Cm^{r}/Amp^{r} phenotype are selected for further study.

Full-length PlyCB is induced from XL1-Blue/pBAD24::plyCB with 0.25% L-arabinose in overnight LB cultures at 37° C. Cells are washed, resuspended in PBS buffer at pH 7.4, and lysed in 20% chloroform to yield crude PlyCB. DNase (100 ug) is added and the suspension and dialyzed in a 6-8 KDa MWCO membrane overnight against 20 mM phosphate buffer, pH 7.4. Next, PlyCB is applied to a ceramic hydroxyapatite (CHT) column (1.5 cm×30 cm) pre-equilibrated with 20 mM phosphate buffer, pH 7.4. All proteins, except PlyCB, where eluted with 200 mM phosphate buffer, pH 7.4. Once the absorbance (A280) had returned to baseline, PlyCB is eluted with 1M phosphate buffer, pH 7.4 as a pure, homogeneous peak. The high molarity phosphate buffer is removed either by concentration and buffer exchange against PBS, or by application to an S-200 gel filtration column pre-equilibrated in PBS and run against the same. Fractions containing PlyCB are confirmed by SDS-PAGE and N-terminal sequence analysis.

PlyCB Self Assembles Into Octamer—Individually expressed PlyCB elutes from gel filtration at approximately 64 kDa, suggesting that it self assembles into an octamer. When gel filtration is repeated with 0.1% SDS, PlyCB elutes at approximately 8 kDa, the mass of the monomer.

FIG. 3 depicts three Superose 12 gel filtration runs superimposed on each other. The blue line shows the elution profile of Bio-Rad gel filtration standards, with masses displayed above each peak. The red line shows the data for purified PlyC holoenyzme, which elutes at ˜114 kDa (1×50 kDa PlyCA+8×8 kDa PlyCB). Purified PlyCB elutes as a single, homogeneous peak at ˜64 kDa. The inset shows an SDS-PAGE from fractions of the PlyC peak (red lane) and PlyCB peak (green lane), indicating purity of the sample and presence of the expected subunit(s).

Upon heating to 50 degrees C., PlyCB retains its three-dimensional structure. PlyCB may be frozen, lyophilized, or stored at 4 degrees C. for one year without any deleterious effects.

In the presence of 8 M urea, PlyCB is denatured into single 8 kDa monomers. Subsequent dialysis to remove the urea results in the refolding of the 64 kDa octamer. Heteromers are formed by engineering a variant of the monomer and mixing the variant monomer with wild-type monomers.

### Example 2 Crystal Structure of PlyCB

Crystallisation of PlyCB—Crystals of the PlyCB octamer are obtained using the hanging drop vapour diffusion method. 0.5 μl of protein solution (10 mg/ml) and 0.5 μl of reservoir buffer (various buffer conditions) are mixed and allowed to equilibrate over 0.5 ml of reservoir buffer at 4° C. Crystals are mounted in nylon cryo loops before being flash-frozen in a nitrogen stream at 100 K. No additional cryo-protectants are used.

Data Collection, structure determination and refinement—Unless otherwise stated all programs used for structural and crystallographic analysis are located within the CCP4 interface (Potterton et al., 2003) to the CCP4 suite (CCP4, 1995). X-ray diffraction data are measured in house, using a Rigaku RU-H3RHB rotating anode X-ray generator equipped with osmic mirrors and a Rigaku R-Axis IV⁺⁺ image plate detector. Data collection is carried out at 100 K. The data are processed and scaled using the packages MOSFLM and SCALA (Evans, 2006; Leslie, 1992).

PlyCB is solved, to 1.7 Å, Rfree 21% Rfactor 20%. The first 9 amino acids in the N-terminus are disordered as are the last two in the C-terminus. Phasing is performed by autoSHARP followed by ARP/WARP and manual building/refinement. Met-70 is used for sulphur single-wavelength anomalous dispersion (S-SAD) analysis.

Structural analysis—PyMOL is used to prepare FIGS. 4, 5, and 6 (Delano, 2002). The crystal structure of PlyCB reveals a relatively flat ring-like structure comprising eight polypeptides. See FIGS. 4 and 5. The ring-like structure is about 16 angstroms tall and about 40 angstroms in diameter giving the protein two distinct faces. The PlyCB monomer is made up of four-strands and two-helices and are numbered sequentially from the N-terminus as shown in FIG. 6. The multiple intermolecular contacts between the -helices of neighboring PlyCB monomers (FIG. 4) are thought to provide considerable stability to the octamer. The crystal structure of PlyCB further reveals a central pore of approximately 2 nm. CCP4MG is used to calculate the electrostatic surface of PlyCB shown in FIG. 5 (Potterton et al., 2004). A surface electrostatic potential map (FIG. 5) shows a strong positive charge on the bottom face and a neutral charge on the top face.

### Example 3 PlyCB Mutants

Site-Directed Mutagenesis—Specific mutations to plyCB within the plyC operon are made by using the QuikChange site-directed mutagenesis kit (Stratagene) according to the manufacturers instructions. The plasmid inserts are digested out of the resulting clones and recloned into pBAD24, and the full-length inserts are sequenced to confirm that only the desired nucleotide sequence changes had been introduced. Expression of point mutants and wild-type PlyCB are carried out in 0.5% arabinose, as described above.

Streptococcal-Binding Properties—Five hundred micrograms each of purified of PlyCB is reacted with the carboxylic acid, succinimidyl ester of Alexa Fluor 568, or Alexa Fluor 488 (Molecular Probes) according to the manufacturerλs instructions. Unreacted dye is removed from the labeled protein by application to a 5-ml HiTrap Desalting column (GE Healthcare), and equilibrated with PBS. Streptococcal cultures are grown overnight, washed in PBS, mixed with 50 g of labeled PlyCB, washed two times in PBS, and mounted on a slide. An Eclipse E400 microscope with a Plan Apo X100 objective (both from Nikon) is used, and images are obtained by using a Retiga Exi camera with Q CAPTURE PRO software (both from Qlmaging, Burnaby, BC, Canada).

N-terminal Deletion—The Del-8 mutant lacks the first 8 N-terminal amino acids of PlyCB but retains full binding activity. Formation of a stable octamer is confirmed by comparing the elution profile of the Del-8 PlyCB mutant on a Superose 12 gel to Bio-Rad gel filtration standards. Accordingly, the N-terminal region can be completely deleted and not affect formation of the stable octamer. Given the non-structural role of this sequence, this region can be modified or engineered.

Loop Region Mutants—The mutations D21A and D21N involve the substitution of aspartic acid for alanine and asparagine respectively. The mutation K23A involves the substitution of lysine for alanine. Each of these mutations is in the loop region between -strands 1 and 2 encompassing D₂₁G₂₂K₂₃E₂₄S₂₅ (see FIG. 6) and retains their ability to bind streptococcal cells. Formation of a stable octamer is tested by comparing the elution profile of the PlyCB mutants on a Superose 12 gel to Bio-Rad gel filtration standards.

RGD Mutants—The peptide sequence RGD is introduced into the external loop of PlyCB. RGD is known to directly bind to cell lines bearing one of the integrins associated with angiogenesis and cancer metastasis (v3, v5 or 51). The GKE-RGD mutant is formed by substituting RGD for G₂₂K₂₃E₂₄ in the wild-type PlyCB (SEQ ID NO. 3) or G₁₄K₁₅E₁₆ in the Del-8 PlyCB mutant (SEQ ID NO. 4). The KES-RGD mutant is formed by substituting RGD for K₂₃E₂₄S₂₅ in the wild-type PlyCB (SEQ ID NO. 5) or K₁₅E₁₆S₁₇ in the Del-8 PlyCB mutant (SEQ ID NO. 6). Both mutants, GKE-RGD and KES-RGD are purified on a hydroxyapatite column. The mutants retain their ability to bind to streptococcal cells. Formation of a stable octamer is tested by comparing the elution profile of the RGD PlyCB mutants on a Superose 12 gel to Bio-Rad gel filtration standards.

Calu-3-Binding Properties—The mutants are also cross-linked with Alexa-Fluor 488. Unreacted dye is removed from the labeled protein by application to a 5-ml HiTrap Desalting column (GE Healthcare), and equilibrated with PBS. The number of florescein molecules per PlyCB is calculated from absorbance sprectroscopy. Calu-3 cells, a human lung carcinoma cell line known to express v3, are grown on glass coverslips. Calu-3 cells expression of the target receptor v3 integrin is verified by immunofluorescence utilizing a fluorescein-conjugated anti-v3 mAb. The Calu-3 cells grown on coverslips are incubated with the PlyCB RGD mutants in serum-free medium for 30 min at 37° C. in a 5% CO2 incubator. The fluorescein concentration of the PlyCB-fluorescein preparations is normalized to 2.5 m to facilitate comparison. After incubation, the cells are washed five times with DPBS, fixed with 4% paraformaldehyde for 10 min, washed with DPBS, and then mounted on slides. Illumination intensity and camera exposure time are held constant. As can be seen in FIG. 7, the RGD mutants tested are sufficient to direct binding to Calu-3 cells.

Flow cytometry is performed on a FACSCalibur (Becton Dickinson, Mountain View, Calif.) and analyzed with Cell Quest software (Becton Dickinson). Adherent Calu-3 cells are nonenzymatically disassociated from cell culture dishes with DPBS without Ca²⁺ or Mg²⁺ plus 1% ethylenediaminetetraacetic acid (EDTA; 25 mM) (for about 2 min at room temperature), washed once with serum-containing medium, and finally suspended in DPBS plus Ca²⁺/Mg²‘at 2.1×10⁶ cells/ml. Experiments are carried out both in the presence and absence of 1% FBS in the buffer. Fluorescently labeled PlyCB preparations (normalized to 2 mM fluorescein) are incubated with cells on ice from 20 min to 2 hr. After incubation, the cells are washed five times with DPBS (both with and without Ca2+/Mg2+), and suspended in DPBS plus 1% FBS for FACS analysis; 10,000 events are counted for each condition. Both the anti-v3 mAb (LM609) (Chemicon MAB1976Z) and the corresponding fluorescein-conjugated anti-v3mAb (Chemicon MAB1976F) are used for FACS analysis.

RGD mutants are also formed by covalently binding the RGD sequence directly or via a linker sequence to the N-terminus of SEQ ID NO:2 and tested as described above.

Other Integrin-Binding Mutants—Various publications have suggested the following sequences also bind integrins associated with angiogenesis and cancer metastasis such as v3, v5 or 51: DGR (the inverse of RGD), REDV, NGR, NGRAHA, RCDVVV, KGD, and ECD. Moreover, it has been reported that peptide CDCRGDCFC, which forms ring through disulfide bonding of the cysteines, has a higher affinity for integrins than a linear RGD peptide. It is likely that the inverse of these peptides will also bind to such integrins (e.g., VDER, RGN, AHARGN, VVVDCR, DGK, ECD, and CFCDGRCDC). These sequences are substituted for some or all of the amino acids 21-25 of SEQ ID NO. 1 or 13-17 of SEQ ID NO. 2. Formation of a stable octamer is tested by comparing the elution profile of the PlyCB mutants on a Superose 12 gel to Bio-Rad gel filtration standards. The mutants are cross-linked with Alexa-Fluor 488 and tested for their ability to bind to Calu-3 cells as described above.

### Example 4 Methionine Analog Labeling of PlyCB

E. coli B834 cells are transformed with pBAD24::plyCB and methionine auxotrophy is confirmed by observing growth in M9 minimal media supplemented with 50 g ml⁻¹ methionine and no growth in M9 media alone. A fresh 20 ml overnight culture of B834/pBAD24::plyCB is washed twice with M9 media, and used to inoculate a 2 L flask of M9 minimal media supplemented with 40 mg/L (0.3 mM) of a methionine analog. Expression of the methionine analogs containing PlyCB is induced with 0.5% arabinose and the PlyCB purified as described above.

### Example 5 Preparation of Contrast Reagent

PlyCB is expressed recombinantly in E. Coli in the arabinose-inducible expression pBAD24 (Amp^{r}) vector and purified as described above. Yields of up to 50 mg of PlyCB per liter of culture are obtained.

Lysine-reactive DTPA-ITC (2-(4-isothiocyanatobenzyl)-diethylenetriamine-pentaacetic acid) is used to chelate a lanthanide such as gandolinium to PlyCB or one of the PlyCB integrin-binding mutants discussed above. Because PlyCB monomer has seven lysine residues that could bind a Gd-DTPA-ITC, there are 56 potential binding sites on PlyCB. The chemical reactivity of individual amino acid residues of PlyCB depends on the microenvironment at each specific site. To estimate the extent of reactivity of the seven lysine residues per monomer, PlyCB is labeled with fluorescein isothiocyanate (FITC). PlyCB protein concentrations are determined through amino acid analysis. Fluorescein modification yields a maximum number of fluorescein groups per PlyCB. Polyvalency, or extent of labeling, is determined by lysine labeling of PlyCB with fluorescein isothiocyanate (FITC) and then measuring fluorescent intensity or directly determining increased mass of the monomer due to the label by mass spectrometry. Alternately, the mass spectrometry approach is used with Gd-DTPA-ITC.

The PlyCB(Gd-DTPA-ITC) contrast reagent is synthesized through the conjugation of pre-metalated Gd-DTPA-ITC (a 300-fold molar excess to PlyCB) to lysine residues. Covalent conjugation is evidenced by a shift in electrophoretic mobility. Individual monomers migrate more slowly during denaturing SDS-PAGE because of the addition of Gd-DTPA-ITC to the PlyCB monomer.

To evaluate the utility of these nanoparticles as MRI contrast agents, relaxivity is determined at two magnetic field strengths. The samples are imaged in a 96-well plate on a 1.5 T Siemens clinical magnet using a multichannel head coil. Relaxivity is also determined at 9.4 T on a Bruker AV 400 MHz narrow-bore NMR spectrometer using a standard 5 mm quartz tube.

To assess the relative visual signal at 1.5 T, a T1-weighted image is acquired in transverse section through the plate. Subsequently, 2D-inversion recovery (IR) images are acquired using a TR/TE of 10 000/21 ms and a T1 of 50-8000 ms (25 values). T1 fitting is performed using magnitudes from the IR fast spin-echo data on standardized regions of interest. Relaxivities for each sample are calculated using the T1 data and gadolinium concentrations as determined by ICP analysis. Magnevist® is used as a positive control. Unlabeled PlyCB soaked in Magnevist® served as negative controls.

### Example 6 Implant Coating

This Example utilizes the unique hydroxyapatite binding properties of PlyCB with an osteoblast binding epitope (integrin-binding) identified above.

Directional Binding of Hydroxyapatite—The directional binding of PlyCB to hydroxyapatite (HA) is determined. A series of monoclonal antibodies are generated and mapped to identify those that can differentiate the top face of PlyCB from the bottom face. These monoclonal antibodies are then used to probe PlyCB that has been bound to a glass cover slip coated in HA. Alternately, PlyCB is viewed with transmission electron microscopy to determine directional binding. The binding affinity of PlyCB for HA is determined by coating a gold sensor chip with hydroxyapatite and determining binding constants with a Biacore® system (Biacore AB).

PlyCBλs ability to bind to osteoblasts while adhered to HA is tested. PlyCB is bound to HA coated cover slips which are then exposed to isolated rodent osteoblasts. Binding is confirmed by light microscopy.

Animal model and titanium implants—A total of twenty 9-wk-old male New Zealand White rabbits are used. Titanium and hydroxyapatite (HA) coated titanium implants are purchased from a commercial vendor. The implants have a screw-type geometric design. The threaded area of the implant consists of a core of 3.5-mm diameter with threads of 0.3-mm height and pitch 0.6 threads/mm. The implants contain a 3-mm abutment that remains non-submerged when implanted.

Adsorption of PlyCB to the implant—PlyCB, or PlyCB-RGD (RGD inserted in external loop as described above) are adsorbed (1 mg/ml) to the HA-implant by a 30 minute soak step. Due to the affinity of PlyCB and PlyCB-RGD to HA, no crosslinkers or chemical reactions are needed. SEM and TEM are used to verify even distribution of the PlyCB label. Alternately, PlyCB antibodies or PlyCB-AlexaFluor conjugates could be used to verify even coating.

Surgical implantation—General anesthesia is induced via intramuscular injection of 45 mg/kg ketamine and 5 mg/kg xylazine. Lidocaine/epinephrine is administered by intramuscular injection into the femoral muscle mass. After skin incision from the lateral side, separation of muscle and reflection of the soft tissue in the proximal femur region, the metaphysis region of the femur is marked with a round bur. Using a twist drill and torque control unit, a pilot hole is drilled, and the diameter of the pilot hole is sequentially increased using drill bits of 2.2, 2.8, and 3.5 mm at speeds of no greater than 800 rpm. The titanium implants are placed transversely and transcortically into the right proximal femur. After replacement of the deflected soft tissue, the skin incision is closed. An identical titanium implant is placed in the corresponding proximal area of the left femur under the same conditions. The implants are allowed to heal for 6 weeks. The rabbits are monitored daily for weight gain and cage behavior. The 20 rabbits are divided into four groups of five:

1. Control, titanium only implant.

2. Control, HA-coated titanium implant.

3. HA-coated titanium implant, PlyCB adsorbed.

4. HA-coated titanium implant, PlyCB-RGD adsorbed.

Tissue harvest and processing—After 6 weeks, the rabbits re euthanized to obtain bone-implant specimens using a surgical saw. The bone block is trimmed into two equal halves along the long implant axis with a diamond saw, with the proximal half to be used for fluorescent microscopy and the distal half to be used for bone histomorphometry. Serial sections are then cut in both directions, further into the bone marrow and externally toward the cortex of the bone. Once trimmed, the bone/implant samples are stored in 4% formalin. Implants are manually removed from all bone specimens with a pair of forceps.

Data analysis—For each sample, one-half of the specimen is decalcified, embedded in paraffin, cut into 5-um sections with microtome, and stained with various histological dyes including hematoxylin and eosin and safranin O/fast green. Bone histomorphometric parameters are quantified using computerized image analysis. Bone in-growth is quantified by static histomorphometry of bone immediately adjacent to implants. Bone density 1 mm from the implant-bone interface (BV/TV) is selected so that bone density near the implant would be quantified. The number of osteoblast-like cells per bone surface (NOb/BS) are measured. BV/TV and NOb/BS are measured on hematoxylin and eosin-stained sections. Safranin O stains for glycosaminoglycans in the extracellular matrix produced by cartilaginous and fibrous tissue is used to detect whether any chondrogenic cells might be present since cartilage and fibrocartilage is known to be present transiently during bone healing. The other half of bone specimen is used for undecalcified preparation, embedded in methyl-methacrylate, sectioned at 12-um thickness with Leica Polycut (SM2500E, Leica), and used for quantification of dynamic bone histomorphometry by fluorescent microscopy.

### Example 7 Quantum Dot and Nanogold arrays

Site-directed mutagenesis—The S1C mutation to plyCB is made using the QuikChange Site-Directed Mutagenesis Kit (Stratagene) according to the manufacturers instructions. The following primers are used: S1C-F, 5λ-GGT AAA ATC AAA TGT GCA AGA TTA ATG TAA ACG TAG AAA ATG TTT CTG G-3λ and S1C-R, 5λ-CCA GAA ACA TTT TCT ACG TTT ACA TTA ATC TTG CAC ATT TGA TTT TAC C-3λ The plasmid inserts are digested out of the resulting clones, re-cloned into pBAD24, and the full length inserts are sequenced to confirm that only the desired nucleotide sequence changes had been introduced. Expression of PlyCB-S1C and wild-type PlyCB are carried out in 0.25% arabinose as previously described.

PlyCB assembly and crystallization—The PlyCB octamer is assembled from purified monomers with the concomitant formation of 2D crystals in solution, without the need for an interacting interface. 5 mM TCEP is used to keep single cysteine residue per monomer in a reduced state. The crystallization solution is incubated at 4C overnight, after which crystals are observed as a white precipitate.

QD array formation—Crystalline PlyCB protein templates are applied to formvar-coated 200 mesh copper TEM grids and QDs (Ted Pella) are bound by floating the sample side of the grid on 5 μl drops of passivated QD sols, wicking away with filter paper and washing by floating on HAT buffer, 25 mM HEPES, 0.1% sodium azide, 3 mM TCEP, pH 7.5, for 10 minutes. Samples are viewed in a LEO 912 AB TEM at 60 kV. All quantitative image analysis is performed using AnalySIS 3.5 (Soft Imaging System).

Nanogold arrays are fabricated in the following manner. Monomers of the SIC mutant are immobilized on either a negative charged glass slide or a hydroxyapatite coated glass slide. The array is then reacted with an excess of Nanogold (monomaleimido Nanogold, Nanoprobes) as per the instructions supplied by the manufacturer, which will react with the thiol group of the sole engineered cysteine residue. The Nanogold-tagged monomers are separated from unreacted protein and excess Nanogold using gel filtration chromatography (BioRad BioGel P-10), concentrated to 1.5 mg/ml and assembled into the octameric rings and 2D crystals as described above.

Analytical electron microscopy (AEM) measurements. Samples are applied to carbon-coated grids (Ted Pella) that are briefly treated with an oxygen plasma to enhance protein adhesion to carbon. Specimens are analyzed at room temperature using a double-tilt Be stage in a FEI TecnaiF20 AEM. The instrument is operated in the TEM, STEM, HAADF EFTEM modes at 200 kV using a Schottky field-emisson gun electron source.

### Example 8 Selective Attachment and Release of a Chemotherapeutic Agent from the Interior of PlyCB

Introduction of cysteine residue—A SIC mutation or other cysteine substitution to PlyCB is made as discussed in Example 7 above.

Linkage of chemotherapeutic agent—A (6-maleimidocaproyl) hydrazone derivative of doxorubicin18 (Mal-Dox) is linked to PlyCB via coupling of the maleimide group from Mal-Dox and the thiol group from the cysteine residues. PlyCB are reacted with an excess (3 molar equivalents) of Mal-Dox in HEPES (100 mM, pH 6.5) for 1 hour at room temperature. Immediately following the reaction, derivatized PlyCBs are separated from free Mal-Dox by size exclusion chromatography (Superose 6, Amersham-Pharmacia). Coelution of PlyCB and doxorubicin after 21 min indicates that doxorubicin is associated with PlyCB. Both transmission electron microscopy (TEM) and dynamic light scattering analysis confirm that PlyCB-Dox maintains the 4 nm diameter of underivatized PlyCB.

The covalent linkage of doxorubicin to PlyCB monomer is demonstrated by SDS-polyacrylamide gel electrophoresis (SDS-PAGE). Fluorescence imaging of an SDS-PAGE gel is used to reveal a mobility shift of doxorubicin (excitation 488 nm; emission 520 nm) bound to PlyCB monomer compared to that of free doxorubicin. Subsequent staining of the protein bands with Coomassie blue is used to illustrate the co-migration of the PlyCB monomer band with the fluorescent (doxorubicin) band indicating their covalent linkage.

Covalent attachment of doxorubicin to all 8 PlyCB monomers is confirmed by liquid chromatography/electrospray mass spectrometry (LC/MS) analysis. PlyCB-Dox is injected onto a C8 column and eluted with a H2O‘acetonitrile gradient; both solvents contained 2% acetic acid at pH 2.3. Analysis of the single LC peak by electrospray mass spectrometry is used to detect two protein components. Deconvolution of the electrospray mass spectrum is used to detect both the complete PlyCB monomer+linker+doxorubicin and PlyCB+linker without the doxorubicin. The detection of only two protein components, at similar concentrations, is indicative of complete labeling of PlyCB with Mal-Dox.

Selective release of doxorubicin—The selective release of doxorubicin from the PlyCB octamer through hydrolysis of the hydrazone linkage under acidic conditions. Doxorubicin release studies are performed at pH 4.0, 4.5, and 5.0. After incubation for times ranging from 0.25 to 24 h, doxorubicin labeled PlyCB is separated from free doxorubicin by chromatography. The amount of doxorubicin that remained associated with the PlyCB octameric protein is quantified by absorbance spectroscopy. Such date is used to show the potential for doxorubicin release from the octameric protein under biologically relevant (lysosomal) conditions.

### Example 9 Measuring Changes in Conductance Across plyCB Channel Caused by Sequential Passage of ss-DNA

Single channels are formed in a horizontal bilayer of diphytanoyl phosphatidylcholine by using PlyCB. The horizontal bilayer is formed across a 30-mm conical Teflon aperture at one end of a Teflon tube. PlyCB is added to the cis chamber on one side of the bilayer. Eight PlyCB monomers assemble to form a large aqueous pore ca 16 Å long and 20 Å in diameter. The cis chamber, to which the samples of DNA are added, accommodated tubing connections that allowed complete chamber flushing. The entire apparatus is mounted on a custom made temperature-controlled base utilizing a thermoelectric device that maintained the buffer solution at a fixed temperature. The setup is enclosed in a grounded copper box to provide electrical shielding and to minimize evaporative solution loss by maintaining a high water vapor pressure atmosphere.

With the cis side negative, 120 mV is applied across the channel. The resultant ionic current flow through the PlyCB channel is amplified and measured by using a patch-clamp amplifier and head-stage. The amplified signals are low-pass filtered at 100 KHz (3302 filter, Krohn-Hite, Avon, Mass.) and digitized at 333 KHz with a 12-bit analogy digital board (Axon).

Single-stranded DNA polymers are purchased from GenWiz and purified by HPLC. The concentration of the DNA is estimated from the absorbance at 260 nm after being redissolved in 1 mM TE buffer. In a typical experiment, single-stranded DNA is added to the cis chamber to a final concentration of 500 nM.

Translocation events are defined as those that decreased the current to less than 30% of the open channel current. Setting this current ratio as the threshold in the acquisition software (CLAMPEX 7, Axon) avoided recording most of the short partial blockades that are proposed to be associated with DNA molecules colliding with, but not fully translocated through, the pore. The stored data are analyzed with custom software that calculated the duration time and average translocation current for each event.

### REFERENCES

Altschul et al. (1990), J. Mol. Biol. 215:403-410.
Altschul et al. (1997) Nucleic Acids Res. 25:3389-3402. Ashkenasy, N., Sanchez-Quesada, J., Bayley, H. and Ghadiri, M. R. (2005) Angew. Chem. Int. Ed. 44, 1401-1404.
CCP4. (1994) Acta Crystallogr. D Biol. Crystallogr. 50, 760-763.
Chevalier, B. S. & Stoddard, B. L. (2001) Nucleic Acids Res. 29, 3757‘ 3774.
Clark, P. A., Rodriguez, A., Sumner D. R., Hussain, M. A., Mao, J. J. (2005). Modulation of bone ingrowth of rabbit femur titanium implants by in vivo axial micromechanical loading. J Appl Physiol 98: 1922‘ 1929.
Dayhoff, M. O., Schwartz R. M. & Orcutt, B. C. (1978). In Atlas of Protein Sequence and Structure (Dayhoff, M. O., ed.). 5(3):345-352, Natl. Biomed. Res. Found., Washington, D.C.
DeLano, W. L. (2002) The PyMOL User's Manual San Carlos, Calif., USA.
Evans, P. (2006) Acta Crystallogr. D Biol. Crystallogr. 62(1), 72-82.
Fischetti, V. A., Jones, K. F. & Scott, J. R. (1985) J. Exp. Med. 161, 1384‘ 1401.
Fischetti, V. A. (2005) Trends Microbiol. 13, 491‘ 496.
Flenniken, M. L., Liepold, L. O., Crowley, B. E., Willits, D. A., Young, M. J., and Douglas, T. (2005). Chem. Commun. (Camb.) 447‘ 449.
Karlin and Altschul (1990) Proc. Natl. Acad. Sci. USA 87:2264-2268.
Karlin and Altschul (1993) Proc. Natl. Acad. Sci. USA 90:5873-5877.
Krause, R. M. (1957) J. Exp. Med. 106, 365‘ 384.
Leslie, A. G. W. (1992) Newsletter on protein crystallography 26.
Loeffler, J. M., Nelson, D. & Fischetti, V. A. (2001) Science 294, 2170‘ 2172.
Loessner, M. J. (2005) Curr. Opin. Microbiol. 8, 480‘ 487.
Matsudaira, P. (1987) J. Biol. Chem. 262, 10035‘ 10038.
McMillan, R. A., Paavola, C. D., Howard, J., Chan, S. L., Zaluzec, N. J., and Trent, J. D. (2002). Ordered nanoparticle arrays formed on engineered chaperonin protein templates. Nature Materials 1:247-252.
Meller, A., Nivon, L., Brandin, E., Golovchenko, J., and Branton, D. (2000) Rapid nanopore discrimination between single polynucleotide molecules. P.N.A.S. 97:1079-1084.
Myers and Miller (1988) CABIOS 4:11-17.
Nelson, D., Loomis, L. & Fischetti, V. A. (2001) Proc. Natl. Acad. Sci. USA 98, 4107‘ 4112.
Nelson, D., Schuch, R., Zhu, S., Tscherne, D. M. & Fischetti, V. A. (2003) J. Bacteriol. 185, 3325‘ 3332.
Nelson, D., Schuch, R., Chahales, P., Zhu, S., & Fischetti, V. A. (2006) Proc. Natl. Acad. Sci. USA 103, 10765-10770.
Potterton, E., Briggs, P., Turkenburg, M., and Dodson, E. (2003) Acta Crystallogr. D Biol. Crystallogr. 59(7), 1131-1137.
Potterton, L., McNicholas, S., Krissinel, E., Gruber, J., Cowtan, K., Emsley, P., Murshudov, G. N., Cohen, S., Perrakis, A., and Noble, M. (2004) Acta Crystallogr. D Biol. Crystallogr. 60(12-1), 2288-2294.
Schagger, H. & von Jagow, G. (1987) Anal. Biochem. 166, 368‘ 379.
Schuch, R., Nelson, D. & Fischetti, V. A. (2002) Nature 418, 884‘ 889.
van Hest, J. C. M., and Tirrell, D. A. (1998) FEBS Letters. 428, 68-70.
Wheeler, J., Holland, J., Terry, J. M. & Blainey, J. D. (1980) J. Gen. Microbiol. 120, 27‘ 33.
Xie, J. and Schultz, P. (2006) Nature Reviews. 7, 775-782.
Young, R. (1992) Microbiol. Rev. 56, 430‘ 481.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description.

All patents, applications, publications, test methods, literature, and other materials cited herein are hereby incorporated by reference.

What is claimed:

1. A purified octameric protein comprising eight polypeptides, wherein each of said polypeptides comprises SEQ ID NO:2.

2. The purified octameric protein of claim 1, wherein said eight polypeptides self-assemble into an octameric ring defining a central pore of about 2 nm in diameter.

3. The purified octameric protein of claim 1, wherein said octameric ring comprises a top face that is neutral and a bottom face that is positively charged.

4. A purified octameric protein comprising eight polypeptides, wherein each of said polypeptides comprises a sequence at least 80% identical to SEQ ID NO:2, and said eight polypeptides self-assemble into an octameric ring.

5. A purified octameric protein comprising eight polypeptides, each of said polypeptides comprises SEQ ID NO:2, at least one of said polypeptides has a cellular-binding sequence of 3-9 amino acids substituted for some or all of amino acids 13-17 of SEQ ID NO:2 or covalently bound directly or via a linker sequence to the N-terminus of SEQ ID NO:2, and said eight polypeptides self-assemble into an octameric ring.

6. The purified octameric protein of claim 5, wherein said cellular-binding sequence is an integrin-binding sequence.

7. The purified octameric protein of claim 6, wherein said integrin-binding sequence is selected from the group consisting of RGD, DGR, REDV, VDER, NGR, RGN, NGRAHA, AHARGN, RCDVVV, VVVDCR, KGD, DGK, EDC, CDE, CDCRGDCFC, and CFCDGRCDC.

8. The purified octameric protein of claim 7, wherein said integrin-binding sequence is RGD.

9. The purified octameric protein of claim 8, wherein each of said polypeptides comprises SEQ ID NO:4.

10. The purified octameric protein of claim 9, wherein each of said polypeptides comprises SEQ ID NO:3.

11. The purified octameric protein of claim 8, wherein each of said polypeptides polypeptide comprises SEQ ID NO:6.

12. The purified octameric protein of claim 11, wherein each of said polypeptides comprises SEQ ID NO:5.

13. A contrast reagent for use with magnetic resonance imaging (MRI) comprising
an octameric protein comprising eight polypeptides, each of said polypeptides comprises SEQ ID NO:2, said eight polypeptides self-assemble into an octameric ring having an outwardly facing surface and an inwardly facing surface; and
a contrast agent bound to said octameric protein.

14. The contrast reagent of claim 13, wherein the contrast agent is a lanthanide.

15. The contrast reagent of claim —further comprising a targeting moiety bound to said octameric protein.

16. The contrast agent of claim 15, wherein the targeting moiety is selected from the group consisting of a cellular-binding sequence, a monoclonal antibody, and a metalloporphyrin.

17. An implant coating comprising
an octameric protein comprising eight polypeptides, wherein each of said polypeptides comprises SEQ ID NO:2, wherein said octameric protein is capable of binding to hydroxyapatite; and
an osteoblast binding agent bound to said octameric protein.

18. The implant coating of claim 17, wherein said osteoblast binding agent is a cellular-binding sequence of 3-9 amino acids substituted for some or all of amino acids 13-17 of SEQ ID NO:2.

19. The implant coating of claim 18, wherein said cellular-binding sequence is selected from the group consisting of an integrin-binding sequence, a heparin-binding sequence, or a sequence derived from collagen or laminin.

20. The implant coating of claim 17 further comprising a bone-repair factor bound to said octameric protein.

21. A bone implant comprising
an implant coated with hydroxyapatite;
the implant coating of claim 17.

22. A complex comprising
an octameric protein comprising eight polypeptides, each of said polypeptides comprises SEQ ID NO:2, said eight polypeptides self-assemble into an octameric ring having an outwardly facing surface, an inwardly facing surface, and an interstitial area; and
one or both of the following:
a targeting agent associated with said octameric protein; and
a functional agent associated with said octameric protein.

23. A method for sequencing a single-stranded nucleic acid comprising
providing a single-stranded nucleic acid comprising a sequence of nucleotides;
providing an octameric protein comprising eight polypeptides, wherein each of said polypeptides comprises SEQ ID NO:2, said octameric protein is fixed in an electrically insulating membrane providing a channel there through;
threading said single-stranded nucleic acid through said octameric protein; and
measuring the changes in the conductance across said membrane by the sequential passage of said nucleotides.


SEQUENCE LISTING (6 sequences)

SEQ ID NO: 1 - protein, 71 aa, Artificial Sequence
  PlyCB monomer
SKINVNVENVSGVQGFLFHTDGKESYGYRAFINGVEIGIKDIETVQGFQQIIPSINISKSDVEAIRKAMKK

SEQ ID NO: 2 - protein, 63 aa, Artificial Sequence
  N-terminal deletion variant of PlyCB monomer
NVSGVQGFLFHTDGKESYGYRAFINGVEIGIKDIETVQGFQQIIPSINISKSDVEAIRKAMKK

SEQ ID NO: 3 - protein, 71 aa, Artificial Sequence
  RGD mutant of PlyCB monomer
SKINVNVENVSGVQGFLFHTDRGDSYGYRAFINGVEIGIKDIETVQGFQQIIPSINISKSDVEAIRKAMKK

SEQ ID NO: 4 - protein, 63 aa, Artificial Sequence
  RGD mutant of PlyCB monomer
NVSGVQGFLFHTDRGDSYGYRAFINGVEIGIKDIETVQGFQQIIPSINISKSDVEAIRKAMKK

SEQ ID NO: 5 - protein, 71 aa, Artificial Sequence
  RGD mutant of PlyCB monomer
SKINVNVENVSGVQGFLFHTDGRGDYGYRAFINGVEIGIKDIETVQGFQQIIPSINISKSDVEAIRKAMKK

SEQ ID NO: 6 - protein, 63 aa, Artificial Sequence
  RGD mutant of PlyCB monomer
NVSGVQGFLFHTDGRGDYGYRAFINGVEIGIKDIETVQGFQQIIPSINISKSDVEAIRKAMKK